(19) United States
(12) Patent Application Publication
(10) Pub. No.: US 2025/0014715 A1
(43) Pub. Date: Jan. 9, 2025

(54) DIETARY INTAKE INFORMATION ACQUISITION DEVICE AND DIETARY INTAKE INFORMATION ACQUISITION METHOD

(71) Applicant: Mitsubishi Electric Corporation, Tokyo (JP)

(72) Inventor: Yoshiki MATSUYAMA, Tokyo (JP)

(73) Assignee: Mitsubishi Electric Corporation, Tokyo (JP)

(21) Appl. No.: 18/889,450

(22) Filed: Sep. 19, 2024

Related U.S. Application Data
(63) Continuation of application No. PCT/JP2022/017684, filed on Apr. 13, 2022.

(51) Int. Cl. G16H 20/60; G06V 40/10; G06V 40/20; G10L 15/22; G16H 10/20

(52) U.S. Cl. CPC G16H 20/60; G06V 40/10; G06V 40/28; G10L 15/22; G16H 10/20; G10L 2015/223

(57) ABSTRACT

A dietary intake information acquisition method includes: inferring a dish or an ingredient to be consumed by a user on the basis of a captured image and creating dietary intake information; creating a question asking what a target dish or a target ingredient is; detecting an action of the user related to the target dish or the target ingredient on the basis of the captured image; determining a question timing from a period in which the action of the user related to the target dish or the target ingredient is detected; outputting question voice output information for outputting the question by voice at the question timing; acquiring an uttered speech of the user in response to the question; performing speech recognition; and reflecting, in the dietary intake information, information regarding the dish or the ingredient which has been obtained from the user answering the question.

### CROSS-REFERENCE TO RELATED APPLICATION

The present application is a continuation of International Application No. PCT/JP2022/017684, filed Apr. 13, 2022, which is incorporated herein by reference in its entirety.

### TECHNICAL FIELD

The present disclosure relates to a dietary intake information acquisition device and a dietary intake information acquisition method for acquiring dietary intake information for identifying a dietary status of a user.

### BACKGROUND ART

In order to provide dietary recommendations, health management, or the like for improving user's dietary life, it is necessary to grasp user's dietary status including dishes or ingredients consumed by the user.

Here, it is a burden for the user to register his/her dietary status by some means. To address such a problem, it is conceivable, for example, to identify user's dietary status by image recognition on the basis of a captured image obtained by imaging the user who is eating. However, there is a limit to identifying the user's dietary status by image recognition. For example, there is a problem of an occurrence of so-called blown-out highlights due to light or a problem of difficulty in recognizing a specific ingredient used in a processed or cooked dish.

In view of this, a technique has been conventionally known for identifying a user's dietary status by asking the user a question by voice about dietary contents, such as “What did you have for breakfast today?”, and receiving an answer from the user to the question by voice (for example, Patent Literature 1).

### CITATION LIST

### Patent Literature

Patent Literature 1: JP 2019-192060 A

### SUMMARY OF INVENTION

### Technical Problem

The related art for identifying the user's dietary status by interaction with a user as described above does not prompt the user to intuitively identify the dish or ingredient that is being asked and to answer without hesitation. For example, in a case where the user ate a plurality of dishes and is asked a question such as “What did you have for breakfast?”, the user may not know which dish is specifically questioned, and may hesitate what to answer. Furthermore, in a case where, for example, the user ate only one dish and this dish includes a plurality of ingredients, the user may not know which ingredient is specifically questioned, and may hesitate what to answer.

The related art cannot ask a question to a user in such a manner that the user can surely answer the dish or ingredient consumed by the user when asking the question by voice to the user about the dish or ingredient that has been consumed by the user. Therefore, there is a problem that information regarding the dish or ingredient consumed by the user cannot be obtained. As a result, it is still not possible to identify the user's dietary status by the related art.

The present disclosure has been made to solve the above-described problems, and an object of the present disclosure is to provide a dietary intake information acquisition device capable of acquiring information regarding a dish or an ingredient consumed by a user by asking a question to the user in such a manner that the user can surely answer the consumed ingredient.

### Solution to Problem

A dietary intake information acquisition device according to the present disclosure includes processing circuitry to perform inferring of a dish or an ingredient to be consumed by a user on a basis of a captured image and perform creation of dietary intake information regarding the dish or the ingredient that has been inferred, to perform creation of, for a target dish or a target ingredient among the dish or the ingredient inferred by the inferring, a question asking what the target dish or the target ingredient is, to perform detection of an action of the user related to the target dish or the target ingredient on a basis of the captured image, to perform determination of a question timing at which the question created by the creation is output from a period in which an action of the user related to the target dish or the target ingredient is detected by the detection, to perform output of question voice output information for outputting the question created by the creation of the question by voice at the question timing determined by the determination, to perform acquisition of an uttered speech of the user in response to the question output by the output by voice on a basis of the question voice output information, to perform speech recognition on the uttered speech acquired by the acquisition, and perform reflection of, in the dietary intake information, information regarding the dish or the ingredient which has been obtained from the user performing answer of the question and which has been specified on a basis of a result of the speech recognition.

### Advantageous Effects of Invention

According to the present disclosure, the dietary intake information acquisition device can obtain information regarding the dish or the ingredient consumed by the user by asking the user a question in such a manner that the user can surely answer the consumed ingredient.

### BRIEF DESCRIPTION OF DRAWINGS

FIG. 1 is a diagram illustrating a configuration example of a dietary intake information acquisition device according to a first embodiment.

FIG. 2 is a diagram illustrating an example of an appearance of a robot equipped with the dietary intake information acquisition device according to the first embodiment.

FIGS. 3A and 3B are diagrams for describing an example of a captured image captured by a first camera and an example of a captured image captured by a second camera in the first embodiment.

FIG. 4 is a diagram illustrating an example of a captured image captured by the first camera in the first embodiment.

FIGS. 5A and 5B are diagrams illustrating an example of dish identification information defined in the first embodiment.

FIG. 6 is a diagram for describing an example of dietary intake information created by a dietary content inferring unit in the first embodiment.

FIG. 7 is a diagram for describing an example of dietary intake information in which information regarding a dish or an ingredient obtained from the user as an answer is reflected by a reflection unit in the first embodiment.

FIG. 8 is a flowchart for describing an operation of the dietary intake information acquisition device according to the first embodiment.

FIGS. 9A and 9B are diagrams illustrating an example of a hardware configuration of the dietary intake information acquisition device according to the first embodiment.

### DESCRIPTION OF EMBODIMENTS

An embodiment of the present disclosure will now be described in detail with reference to the drawings.

### First Embodiment

A dietary intake information acquisition device according to the first embodiment creates information (hereinafter referred to as “dietary intake information”) regarding a dish or an ingredient consumed by a user and stores the information in a storage unit. Specifically, the dietary intake information acquisition device according to the first embodiment infers a dish or an ingredient to be consumed by the user on the basis of a captured image. In a case where the dietary intake information acquisition device determines that there is a dish (hereinafter referred to as “target dish”) or an ingredient (hereinafter referred to as “target ingredient”) to which a question asking what the dish or the ingredient is, for the dishes or the ingredients inferred on the basis of the captured image, the dietary intake information acquisition device outputs a question asking what the target dish or the target ingredient is to the user by voice. The dietary intake information acquisition device determines a timing of outputting the question (hereinafter referred to as “question timing”) on the basis of information regarding the target dish or the target ingredient and an action of the user detected on the basis of the captured image. The dietary intake information acquisition device acquires an answer to the question output by voice at the question timing by user's uttered speech, and reflects the user's answer of what the target dish or the target ingredient is from the acquired uttered speech in the dietary intake information.

In the first embodiment, “dish” refers to food or drink combining and processing ingredients, seasonings, and the like. In addition, “ingredient” is an ingredient of a dish and is included in the dish.

The dietary intake information created and stored by the dietary intake information acquisition device identifies, for example, nutrients consumed by the user and is used for recommendation or health management for improving the user's dietary life.

Note that the dietary intake information acquisition device according to the first embodiment is assumed to be a device that is mainly used in common households and creates dietary intake information regarding a dish or an ingredient consumed by the user at a dining table in common households. That is, the user is assumed to be a resident of a common household in the first embodiment.

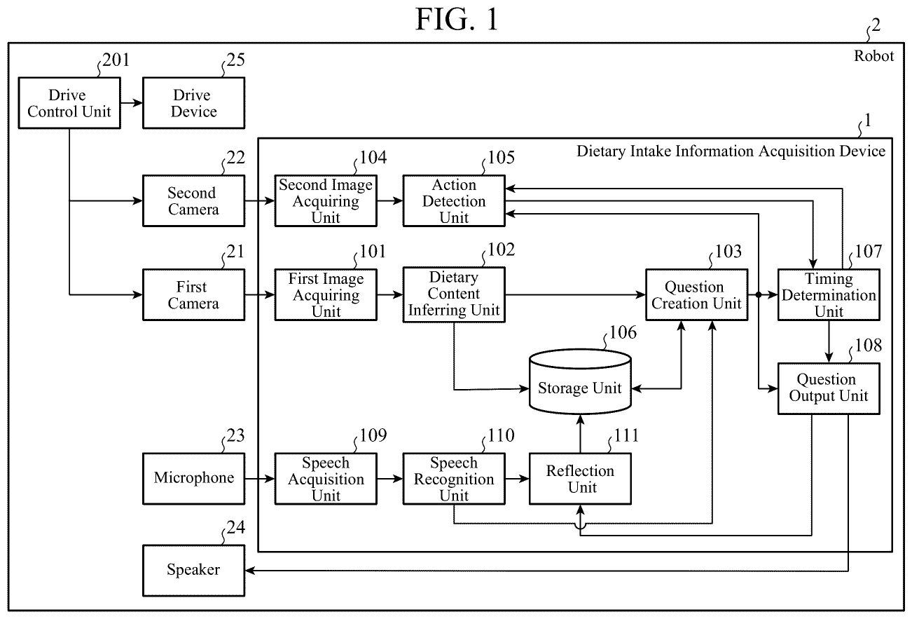

FIG. 1 is a diagram illustrating a configuration example of a dietary intake information acquisition device 1 according to the first embodiment.

The dietary intake information acquisition device 1 according to the first embodiment is assumed to be mounted on a robot 2, for example.

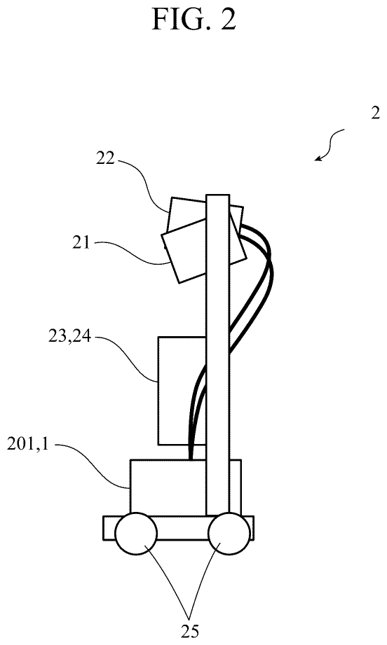

FIG. 2 is a diagram illustrating an example of an appearance of the robot 2 equipped with the dietary intake information acquisition device 1 according to the first embodiment.

The robot 2 includes a drive device 25, and is autonomously movable in a room by the drive device 25. The drive device 25 includes, for example, a plurality of tires, a motor, and the like.

The robot 2 is equipped with a first camera 21 and a second camera 22.

The first camera 21 is provided so as to be able to image at least a dish or an ingredient to be consumed by the user. For example, the first camera 21 is provided so as to be able to image a table at which the user is eating.

The second camera 22 is provided so as to be able to capture an image of the user who is consuming the dish or the ingredient, in other words, who is eating.

Note that FIG. 2 does not illustrate the user, a table at which the user is eating, and a dish, an ingredient, or the like on the table.

The first camera 21 and the second camera 22 include a drive unit (not illustrated) including a motor or the like, and are provided in such a manner that the imaging direction can be changed by the drive unit.

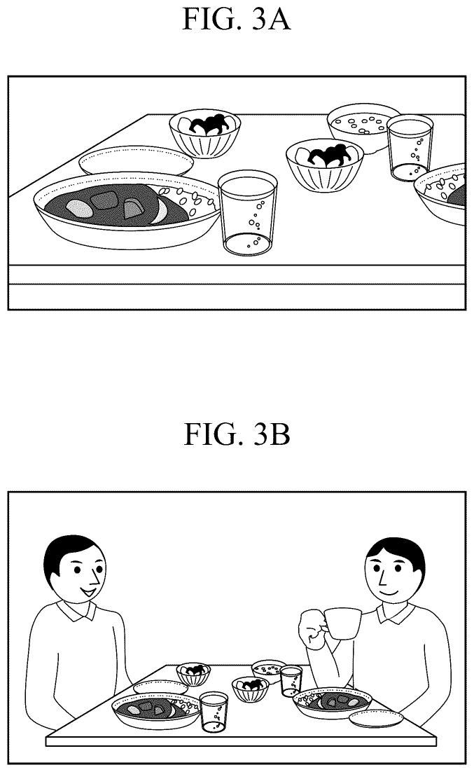

FIG. 3 is a diagram for describing an example of a captured image (hereinafter referred to as “first captured image”) captured by the first camera 21 and an example of a captured image (hereinafter referred to as “second captured image”) captured by the second camera 22 in the first embodiment.

FIG. 3A illustrates an example of the first captured image, and FIG. 3B illustrates an example of the second captured image.

The first captured image includes dishes or ingredients.

The second captured image includes users who are eating, specifically, users who are consuming the dishes or ingredients included in the first captured image.

The robot 2 includes a drive control unit 201 that performs control of the drive device 25, control of the drive unit of the first camera 21, and control of the drive unit of the second camera 22.

The user operates an input device such as a touch panel display (not illustrated) included in the robot 2 to instruct the position of the robot 2, the imaging direction of the first camera 21, and the imaging direction of the second camera 22. The drive control unit 201 moves the robot 2, changes the imaging direction of the first camera 21, or changes the imaging direction of the second camera 22 on the basis of an instruction from the user.

The robot 2 is also equipped with a microphone 23 which is a voice input device and a speaker 24 which is a voice output device.

The microphone 23 collects uttered speeches. The speaker 24 outputs a voice output by the dietary intake information acquisition device 1.

Note that the user can adjust the volume of the voice output from the speaker 24. For example, the user inputs a volume adjustment instruction using the input device included in the robot 2. A volume control unit (not illustrated) included in the robot 2 adjusts the volume of the voice output from the speaker 24 on the basis of the volume adjustment instruction from the user.

As illustrated in FIG. 1, the dietary intake information acquisition device 1 includes a first image acquiring unit 101, a dietary content inferring unit 102, a question creation unit 103, a second image acquiring unit 104, an action detection unit 105, a storage unit 106, a timing determination unit 107, a question output unit 108, a speech acquisition unit 109, a speech recognition unit 110, and a reflection unit 111.

The first image acquiring unit 101 acquires the first captured image captured by the first camera 21.

The first image acquiring unit 101 outputs the acquired first captured image to the dietary content inferring unit 102.

The dietary content inferring unit 102 infers a dish or an ingredient to be consumed by the user on the basis of the first captured image acquired by the first image acquiring unit 101. Then, the dietary content inferring unit 102 creates dietary intake information regarding the inferred dish or ingredient.

When doing so, the dietary content inferring unit 102 calculates degree of certainty indicating the certainty of the inference result of the dish or ingredient.

Note that the dietary content inferring unit 102 may have the function of the first image acquiring unit 101.

In the first embodiment, the first image acquiring unit 101 acquires the first captured image on a frame-by-frame basis. The dietary content inferring unit 102 infers a dish or an ingredient to be consumed by the user on the basis of the latest frame acquired by the first image acquiring unit 101.

A method for inferring a dish or an ingredient to be consumed by the user and a method for calculating degree of certainty of an inference result of the dish or the ingredient by the dietary content inferring unit 102 will be described by way of an example.

For example, the dietary content inferring unit 102 infers a dish or an ingredient to be consumed by the user on the basis of an image recognition result performed using a known image recognition technology, pattern matching, or the like, and dish identification information created in advance by an administrator or the like and stored in a place that can be referred to by the dietary content inferring unit 102.

The dish identification information is, for example, information in which a dish and an ingredient expected to be included in the dish are defined. In the dish identification information, information that can specify a dish is associated with information indicating an ingredient expected to be included in the dish. Note that the information indicating an ingredient may be information that can specify the ingredient itself or may be information that can specify the color or shape of the ingredient.

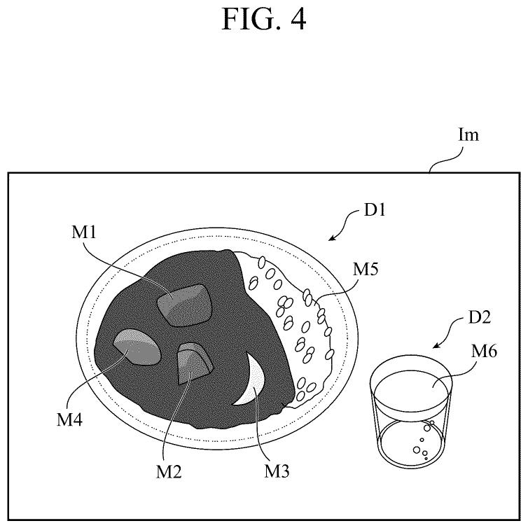

FIG. 4 is a diagram illustrating an example of the first captured image captured by the first camera 21 in the first embodiment. The first captured image is the captured image illustrated in FIG. 4, for example. In FIG. 4, the first captured image is indicated by Im. Further, in FIG. 4, the dish is indicated by D1 or D2, and the ingredients are indicated by M1 to M6. Ingredients included in the dish D1 are ingredients M1 to M5, and an ingredient included in the dish D2 is the ingredient M6.

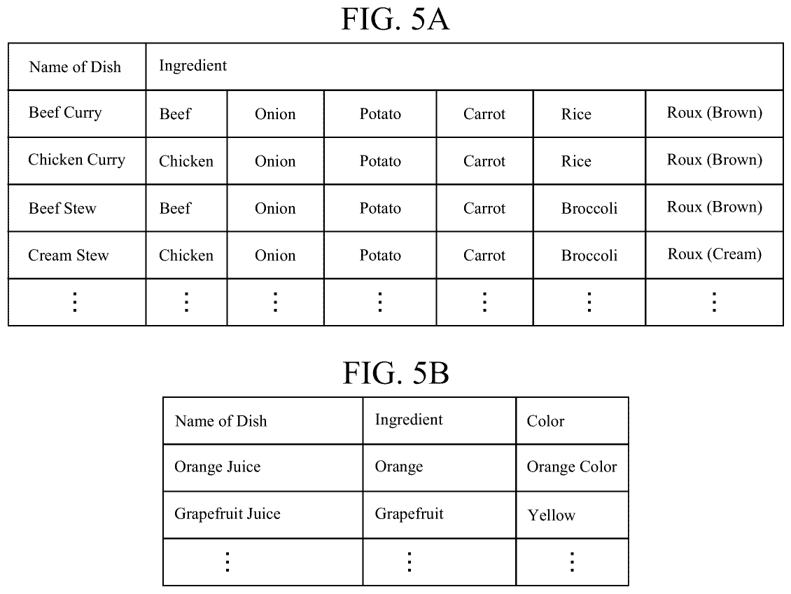

In addition, the dish identification information having contents illustrated in FIGS. 5A and 5B is defined, for example.

For example, the dish identification information illustrated in FIG. 5A is dish identification information related to food, and the dish identification information illustrated in FIG. 5B is dish identification information related to drink.

For example, the dietary content inferring unit 102 first performs known image recognition processing, pattern matching, or the like on the first captured image, and infers the dishes and the ingredients captured in the first captured image.

As a result, the dietary content inferring unit 102 infers that the dish D1 is curry and rice. In addition, the dietary content inferring unit 102 infers the ingredients M1 to M5 as the ingredients included in the curry and rice. Note that the ingredients M1 to M4 are ingredients of roux.

Here, it is assumed that the dietary content inferring unit 102 can infer that the ingredient M5 is rice by image recognition processing or the like on the first captured image. Furthermore, it is assumed that the dietary content inferring unit 102 can infer that the ingredient M3 is onion. On the other hand, it is assumed that the dietary content inferring unit 102 has not been able to infer what the ingredients M1, M2, and M4 are from the image recognition processing or the like on the first captured image, due to the reason that most of the ingredients M1, M2, and M4 are covered with the roux in the first captured image.

Then, the dietary content inferring unit 102 compares the inference result with the dish identification information as illustrated in FIG. 5A.

As a result, the dietary content inferring unit 102 can find that curry and rice includes, for example, beef curry and chicken curry. Moreover, the dietary content inferring unit 102 can find ingredients expected to be included in each of the beef curry and the chicken curry.

Now, the dietary content inferring unit 102 infers that the dish D1 is curry and rice, the ingredient M3 is onion, and the ingredient M5 is rice from the first captured image. However, according to the dish identification information, onion and rice are included in both the beef curry and the chicken curry, and it is not possible to determine whether the dish D1 is the beef curry or the chicken curry only by inferring onion and rice. Therefore, the dietary content inferring unit 102 infers what the ingredients M1, M2, and M4 other than the ingredients M3 and M5 are by referring to the dish identification information on the basis of the first captured image. As a result, the dietary content inferring unit 102 infers that the ingredient M2 looks like carrot. For example, the dietary content inferring unit 102 determines that the ingredient that is inferred to be the closest as the ingredient contained in the curry defined in the dish identification information is carrot from the color, shape, or the like of a slight portion of the ingredient M2 not covered with the roux in the first captured image.

In a similar manner, the dietary content inferring unit 102 infers that the ingredient M1 looks like beef and the ingredient M4 looks like potato.

In addition, the dietary content inferring unit 102 has found, from the dish identification information, that there are, for example, beef curry and chicken curry as curry and rice, and the ingredient for distinguishing the beef curry and the chicken curry is beef or chicken, but has not been able to infer whether the ingredient included in the dish D1 is beef or chicken from the first captured image. However, the dietary content inferring unit 102 has inferred that the ingredient M1 looks like beef, and thus, infers that the dish D1 looks like beef curry.

The dietary content inferring unit 102 also infers what the dish D2 and the ingredient M6 are by a method similar to that for the dish D1 and the ingredients M1 to M5. Here, it is assumed that the dietary content inferring unit 102 can infer that the dish D2 is orange juice and the ingredient M6 is orange from the first captured image without referring to the dish identification information.

After inferring the dish (dishes D1 and D2 in the above example) or the ingredient (ingredients M1 to M6 in the above example) from the first captured image or by referring to the dish identification information on the basis of the first captured image, the dietary content inferring unit 102 calculates the degree of certainty of the inference result of the dish or the ingredient.

Regarding the degree of certainty calculated by the dietary content inferring unit 102, what kind of rule is used to calculate the degree of certainty is determined in advance. For example, a rule is determined that, for a dish or an ingredient that can be inferred from only the first captured image, the degree of certainty is calculated in a range of 70 to 100(%) depending on a matching degree of pattern matching or the like, and for a dish or an ingredient that has been inferred on the basis of the first captured image by referring to the dish identification information, degree of certainty is calculated in a range of 0 to 69(%) depending on, for example, an area that can be identified in the first captured image or depending on a comprehensive matching degree between the dish or the ingredient that can be inferred in the first captured image and the dish or the ingredient defined in the dish identification information.

It is only sufficient that the dietary content inferring unit 102 calculates the degree of certainty of the dish or the ingredient in accordance with a predetermined rule.

In the above example, it is assumed, for example, that the dietary content inferring unit 102 has calculated that the degree of certainty of the inference result obtained by inferring the dish D1 as beef curry is 50(%), the degree of certainty of the inference result obtained by inferring the ingredient M1 as beef is 20(%), the degree of certainty of the inference result obtained by inferring the ingredient M2 as carrot is 30(%), the degree of certainty of the inference result obtained by inferring the ingredient M3 as onion is 100(%), the degree of certainty of the inference result obtained by inferring the ingredient M4 as potato is 20(%), the degree of certainty of the inference result obtained by inferring the ingredient M5 as rice is 100(%), the degree of certainty of the inference result obtained by inferring the dish D2 as orange juice is 90(%), and the degree of certainty of the inference result obtained by inferring the ingredient M6 as orange is 90(%).

Note that the method for inferring a dish or an ingredient to be consumed by the user based on the first captured image and the method for calculating the degree of certainty by the dietary content inferring unit 102 as described above are merely examples. The dietary content inferring unit 102 may use another method to infer the dish or the ingredient to be consumed by the user and calculate the degree of certainty of the inference result of the dish or the ingredient.

For example, the dietary content inferring unit 102 may obtain an inference result of the dish or ingredient to be consumed by the user and the degree of certainty of the inference result using a trained model (hereinafter referred to as “machine learning model”).

The machine learning model uses the captured image as an input, and outputs information regarding the dish or the ingredient to which the degree of certainty is given. The machine learning model is created in advance by an administrator or the like, and is stored in a place that can be referred to by the dietary content inferring unit 102.

After inferring the dish or the ingredient, the dietary content inferring unit 102 creates dietary intake information regarding the inferred dish or ingredient.

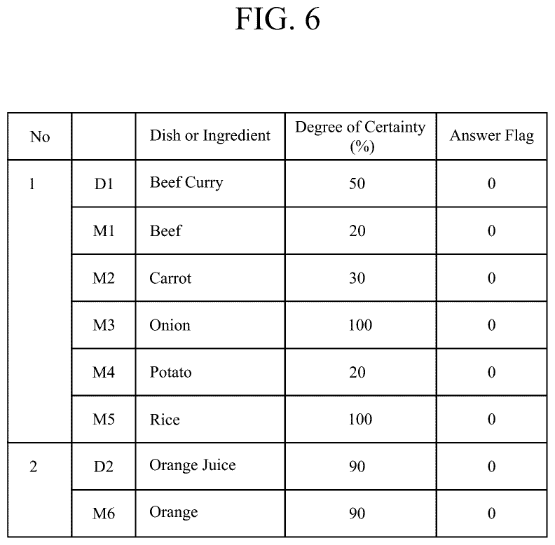

FIG. 6 is a diagram for describing an example of the dietary intake information created by the dietary content inferring unit 102 in the first embodiment.

FIG. 6 illustrates the dietary intake information regarding the dish (D1, D2) and the ingredients (M1 to M6) inferred by the dietary content inferring unit 102 in the example as described with reference to FIGS. 4 and 5.

The dietary intake information is information in which information indicating a dish and information indicating an ingredient are associated with each other. In the dietary intake information illustrated in FIG. 6, the dish and the ingredient corresponding to each other are given the same identification number (“No” in FIG. 6) and are grouped. The dietary intake information includes information that can specify a dish or an ingredient on the first captured image, information indicating the inferred dish or ingredient, degree of certainty, and an answer flag.

In the dietary intake information illustrated in FIG. 6, the information indicating the inferred dish or ingredient is the name of the dish or ingredient, but this is merely an example. It is only sufficient that the dietary content inferring unit 102 sets information from which the inferred dish or ingredient can be found as information indicating the inferred dish or ingredient in the dietary intake information. In addition, in the dietary intake information illustrated in FIG. 6, the information that can specify the dish or the ingredient on the first captured image is D1, D2, and M1 to M6 for convenience, but this is merely an example. It is only sufficient that the dietary content inferring unit 102 sets information that can specify a dish or an ingredient on the first captured image in the dietary intake information. In addition, the dietary content inferring unit 102 adds information (for example, coordinates on the first captured image) indicating the position of the dish or the ingredient in the first captured image to the information that can specify the dish or the ingredient on the first captured image. Note that FIG. 6 does not illustrate information indicating the position of a dish or an ingredient in the first captured image. In addition, the dietary content inferring unit 102 sets the calculated degree of certainty in the dietary intake information. The answer flag will be described later. Furthermore, in the dietary intake information, the first captured image may be associated on an identification-number basis, for example.

The dietary content inferring unit 102 stores the created dietary intake information in the storage unit 106.

In addition, the dietary content inferring unit 102 gives a notice indicating that the dietary intake information has been created to the question creation unit 103. The storage unit 106 stores the dietary intake information.

Note that, in FIG. 1, the storage unit 106 is provided in the dietary intake information acquisition device 1, but this is merely an example. The storage unit 106 may be provided outside the dietary intake information acquisition device 1 at a place that can be referred to by the dietary intake information acquisition device 1.

The question creation unit 103 determines, on the basis of the dietary intake information created by the dietary content inferring unit 102, whether or not there is a target dish or a target ingredient to which a question is to be output among the dishes or ingredients inferred by the dietary content inferring unit 102.

For example, the question creation unit 103 determines that there is a target dish or a target ingredient in a case where there is a dish or an ingredient whose given degree of certainty is less than a preset threshold (hereinafter referred to as “question necessity determination threshold”) on the basis of the dietary intake information stored in the storage unit 106.

The question creation unit 103 determines that there is no target dish and no target ingredient in a case where there is no dish and ingredient whose given degree of certainty is less than the question necessity determination threshold on the basis of the dietary intake information.

Note that the question necessity determination threshold is set in advance by an administrator or the like and is stored in a place that can be referred to by the question creation unit 103. The administrator or the like can set the question necessity determination threshold according to the needs.

In the following, “question target” refers to a target dish or a target ingredient determined by the question creation unit 103 in the first embodiment. That is, in a case where the question creation unit 103 determines that there is a target dish and a target ingredient in the dishes or ingredients inferred by the dietary content inferring unit 102, the question target is the target dish and the target ingredient. In a case where the question creation unit 103 determines that there is only a target dish in the dishes or ingredients inferred by the dietary content inferring unit 102, the question target is the target dish. In a case where the question creation unit 103 determines that there is only a target ingredient in the dishes or ingredients inferred by the dietary content inferring unit 102, the question target is the target ingredient.

When determining that there is the target dish or the target ingredient, the question creation unit 103 creates a question asking what the question target is.

The question creation unit 103 creates, for example, a question asking what the question target is with a demonstrative.

As a specific example, the question creation unit 103 creates a question asking “What is that dish?” or “What is that dish you are eating now?” for the target dish, for example.

Furthermore, the question creation unit 103 creates a question asking “What is that ingredient?”, “What is in that dish?”, or “What is that ingredient you are eating now?”, for example, for the target ingredient.

Note that, in a case where there is a plurality of target dishes or there is a plurality of target ingredients, the question creation unit 103 does not need to create the same question for all the target dishes or all the target ingredients. The question creation unit 103 may create different questions for each of the target dishes, or may create different questions for each of the target ingredients.

The question creation unit 103 creates information regarding the created question (hereinafter referred to as “question information”), and outputs the question information to the action detection unit 105, the timing determination unit 107, and the question output unit 108.

In the question information, a question, information capable of specifying a question target corresponding to the question on the first captured image, and information indicating a position of the question target corresponding to the question on the first captured image are associated. Note that the question creation unit 103 creates question information for each created question, for example.

Here, in a case where there is a plurality of question targets, the question creation unit 103 assigns priority orders to the plurality of question targets in the question information. What kind of rule is used to give priority orders to the plurality of question targets by the question creation unit 103 is determined in advance.

Here, in a case where a certain target dish includes a target ingredient, the question creation unit 103 gives a higher priority order to the target dish than to the target ingredient as an example. Furthermore, in a case where there is a plurality of target ingredients included in a certain target dish, the question creation unit 103 gives a higher priority order to a target ingredient as the target ingredient has lower degree of certainty among the plurality of target ingredients. Note that, in a case where the degree of certainty is the same for a plurality of target ingredients, the question creation unit 103 can optionally set priority orders. In addition, in a case where there is a plurality of target dishes, the question creation unit 103 gives a higher priority order to a target dish as the target dish has lower degree of certainty among the plurality of target dishes. Note that, in a case where the degree of certainty is the same for a plurality of target dishes, the question creation unit 103 can optionally set priority orders.

The second image acquiring unit 104 acquires the second captured image captured by the second camera 22.

The second image acquiring unit 104 outputs the acquired second captured image to the action detection unit 105.

The action detection unit 105 detects an action of the user related to the question target on the basis of the second captured image acquired by the second image acquiring unit 104. The action detection unit 105 specifies the question target from the question information output from the question creation unit 103. The action detection unit 105 may use a known image recognition technology or the like to detect the action of the user.

What kind of action is determined as an action related to a dish or an ingredient by the action detection unit 105 is defined in advance by an administrator or the like.

Note that, due to the installation positions and the angles of view of the first camera 21 and the second camera 22 being determined in advance, the action detection unit 105 can associate the position of the question target on the first captured image with the position of the dish or the ingredient on the second captured image. That is, the action detection unit 105 can associate the question target on the first captured image with a position of the question target on the second captured image. Therefore, the action detection unit 105 can detect an action of the user related to the question target.

For example, the action detection unit 105 detects the user's action of touching a tableware on which the question target is served.

In addition, the action detection unit 105 detects, for example, a user's action of holding the question target with cutlery. In the first embodiment, a tool used by the user when he/she eats a dish or an ingredient is referred to as cutlery. Specifically, the cutlery includes chopsticks, a fork, a knife, a spoon, and the like.

In addition, the action detection unit 105 detects, for example, a user's action of putting the question target into his/her mouth. For example, the action detection unit 105 may detect a user's action of chewing the question target.

In addition, the action detection unit 105 detects, for example, a user's action of swallowing the question target.

The action detection unit 105 outputs information regarding the detected action of the user (hereinafter referred to as “action information”) to the timing determination unit 107. The action detection unit 105 associates, for example, the detected action of the user, information that can specify the question target on which the action has been performed, in other words, the question target related to the action, and information indicating a position (for example, coordinates on the second captured image) of the question target on the second captured image in the action information.

Note that the action detection unit 105 may have the function of the second image acquiring unit 104.

In the first embodiment, the second image acquiring unit 104 acquires the second captured image on a frame-by-frame basis. The action detection unit 105 detects an action of the user related to the question target on the basis of the latest frame acquired by the second image acquiring unit 104.

The timing determination unit 107 determines a question timing at which the question created by the question creation unit 103 is output from the period in which the action detection unit 105 detects the action of the user related to the question target.

Specifically, the timing determination unit 107 determines a time at which the user is performing an action related to the question target as the question timing. For example, the timing determination unit 107 determines a time at which the user is touching the tableware on which the question target is served as the question timing.

Alternatively, the timing determination unit 107 determines a time at which the user is holding the question target with the cutlery as the question timing.

Alternatively, the timing determination unit 107 determines a time at which the user is putting the question target into his/her mouth or a time at which the user is chewing the question target as the question timing.

Alternatively, the timing determination unit 107 determines a time at which the user is swallowing the question target as the question timing.

In a case where there is a plurality of question targets, the timing determination unit 107 specifies which question target is selected as a question target to which the action performed by the user at a time to be determined as the question timing is related in accordance with, for example, the priority orders assigned to the question targets.

The timing determination unit 107 can find the priority order assigned to the question target on the basis of the question information output from the question creation unit 103.

Note that the timing determination unit 107 determines a period during which the user continues the action related to the question target as the question timing.

The timing determination unit 107 may not determine the question timing while, for example, there is a speech by the user or the like. The timing determination unit 107 may determine whether or not there is a speech on the basis of the speech recognition result by the speech recognition unit 110. The speech recognition unit 110 will be described later.

The timing determination unit 107 determines whether or not there is a speech on the basis of the speech recognition result by the speech recognition unit 110 and does not determine the question timing while there is a speech, whereby the dietary intake information acquisition device 1 can prevent an output of a question asking the user what the target dish or the target ingredient is in a situation where the user has difficulty in hearing due to the speech by the user or the like.

When determining the arrival of the question timing, the timing determination unit 107 outputs information (hereinafter referred to as “timing arrival information”) giving a notice indicating the arrival of the question timing to the question output unit 108. The timing determination unit 107 associates information indicating the arrival of the question timing with information indicating which question target the user's action is related to at the time determined as the question timing in the timing arrival information.

The question output unit 108 outputs, to the speaker 24, information (hereinafter referred to as “question voice output information”) for outputting the question created by the question creation unit 103 by voice at the question timing determined by the timing determination unit 107 on the basis of the question information output from the question creation unit 103 and the timing arrival information output from the timing determination unit 107.

Specifically, the question output unit 108 outputs, to the speaker 24, question voice output information for outputting, by voice, a question associated with a question target for which the action of the user, based on which the arrival of the question timing has been determined, is performed in the question information at the question timing determined by the timing determination unit 107. The speaker 24 outputs the question by voice.

The speech acquisition unit 109 acquires surrounding uttered speeches collected by the microphone 23.

The speech acquisition unit 109 acquires the user's uttered speech in response to the question output by voice by the question output unit 108 on the basis of the question voice output information by the question output unit 108.

The speech acquisition unit 109 outputs the acquired uttered speech to the speech recognition unit 110.

The speech recognition unit 110 executes speech recognition processing and performs speech recognition on the uttered speech acquired by the speech acquisition unit 109. The speech recognition unit 110 may use a known speech recognition technology to execute speech recognition processing.

The speech recognition unit 110 outputs the speech recognition result to the reflection unit 111.

The reflection unit 111 reflects, in the dietary intake information stored in the storage unit 106, information regarding a dish or an ingredient which has been obtained from the user answering the question output by voice by the question output unit 108 on the basis of the question voice output information by the question output unit 108 and which has been specified on the basis of the speech recognition result by the speech recognition unit 110.

Note that the question output unit 108 outputs, to the reflection unit 111, information (hereinafter referred to as “output question information”) indicating to which question target the question has been raised when the question voice output information has been output to the speaker 24, for example. The question output unit 108 sets the output question information as, for example, information in which a question and information capable of specifying a question target on the first captured image are associated with each other. The question output unit 108 may further associate information indicating the position of the question target on the first captured image or the first captured image in the output question information. Note that the question output unit 108 can determine information that can specify the question target corresponding to the question on the first captured image and information indicating the position of the question target corresponding to the question on the first captured image on the basis of the question information output from the question creation unit 103. Furthermore, the question output unit 108 may acquire the first captured image from the question creation unit 103. The reflection unit 111 recognizes that a question has been given to the user and the content thereof on the basis of the information output from the question output unit 108.

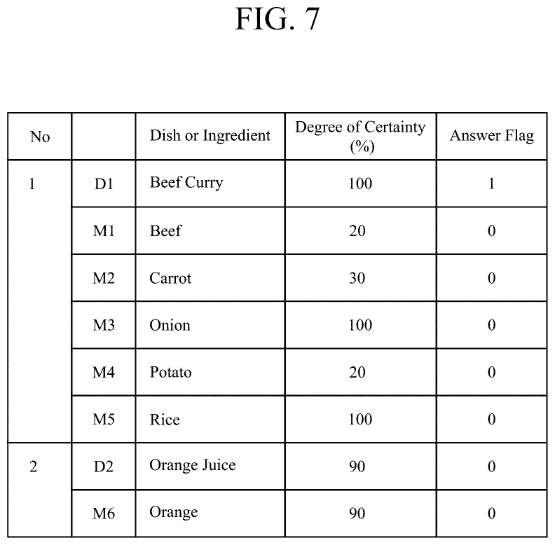

FIG. 7 is a diagram for describing an example of dietary intake information in which information regarding a dish or an ingredient obtained from the user as an answer is reflected by the reflection unit 111 in the first embodiment.

FIG. 7 illustrates an example of the dietary intake information in which a user's answer of “beef curry” is reflected, the user's answer being in response to the question that asks “What is that dish?” about the dish D1 and that is created by the question creation unit 103 on the basis of the dietary intake information as illustrated in FIG. 6, the question being output by voice by the question output unit 108.

The dietary content inferring unit 102 has originally inferred that the dish D1 is beef curry with the degree of certainty of 50(%). In response, the reflection unit 111 determines that the dish D1 is beef curry due to the answer of “beef curry” being obtained from the user. That is, the reflection unit 111 updates the information indicating the dish corresponding to the dish D1 to information indicating beef curry, and updates the degree of certainty assigned to the information indicating beef curry to 100(%) in the dietary intake information.

Note that it is assumed that the user has answered “chicken curry” to the question asking “What is that dish?” for the dish D1. In that case, the reflection unit 111 updates the dietary intake information in such a manner that the information indicating the dish corresponding to the dish D1 is updated to information indicating chicken curry, and reflects the degree of certainty assigned to the information indicating chicken curry as 100(%), for example.

Furthermore, the reflection unit 111 also sets the answer flag attached to the information indicating the updated dish or ingredient in the dietary intake information to “1”. The answer flag indicates that the information indicating the dish or ingredient in the dietary intake information is information indicating the dish or ingredient obtained from the user through the interaction with the user.

The operation of the dietary intake information acquisition device 1 according to the first embodiment will be described.

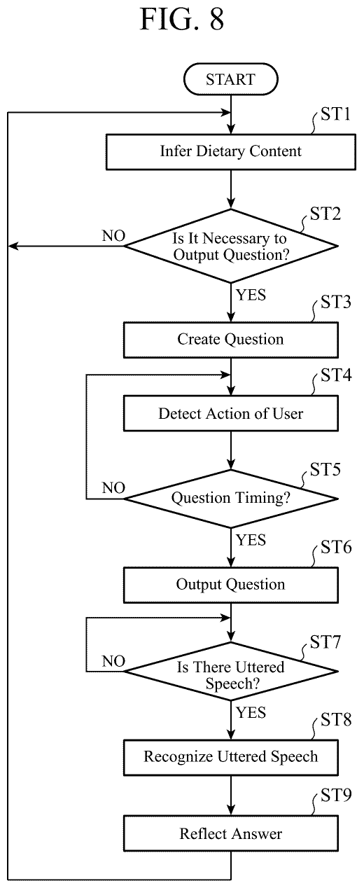

FIG. 8 is a flowchart for describing the operation of the dietary intake information acquisition device 1 according to the first embodiment.

When powered on, the dietary intake information acquisition device 1 starts the operation as illustrated in the flowchart of FIG. 8, for example. Note that this is merely an example, and the dietary intake information acquisition device 1 may start the operation as illustrated in the flowchart of FIG. 8 when, for example, receiving an instruction to start the operation from the user. For example, the user operates the touch panel display included in the dietary intake information acquisition device 1 to input an instruction to start the operation of the dietary intake information acquisition device 1. The dietary intake information acquisition device 1 receives the input instruction and starts the operation.

The first image acquiring unit 101 acquires the first captured image captured by the first camera 21 and outputs the acquired first captured image to the dietary content inferring unit 102.

The dietary content inferring unit 102 infers a dish or an ingredient to be consumed by the user on the basis of the first captured image acquired by the first image acquiring unit 101 (step ST1).

Then, the dietary content inferring unit 102 creates dietary intake information regarding the inferred dish or ingredient. When doing so, the dietary content inferring unit 102 calculates degree of certainty indicating the certainty of the inference result of the dish or ingredient.

The dietary content inferring unit 102 stores the created dietary intake information in the storage unit 106.

In addition, the dietary content inferring unit 102 gives a notice indicating that the dietary intake information has been created to the question creation unit 103.

The question creation unit 103 determines, on the basis of the dietary intake information created by the dietary content inferring unit 102 in step ST1, whether or not there is a target dish or a target ingredient among the dishes or ingredients inferred by the dietary content inferring unit 102 (step ST2).

When the question creation unit 103 determines that there is no target dish and no target ingredient (“NO” in step ST2), the operation of the dietary intake information acquisition device 1 returns to the process of step ST1.

When determining that there is a target dish or a target ingredient (“YES” in step ST2), the question creation unit 103 creates a question asking what the question target is for the question target (step ST3).

As a specific example, it is assumed, for example, that the dietary intake information stored in the storage unit 106 is the dietary intake information having contents as illustrated in FIG. 6. In addition, it is assumed that 70(%) is set as the question necessity determination threshold. In this case, the dish D1, the ingredient M1, the ingredient M2, and the ingredient M4 are less than the question necessity determination threshold, and thus, the question creation unit 103 determines that there is a target dish and a target ingredient.

Then, the question creation unit 103 creates, for each of the dish D1, the ingredient M1, the ingredient M2, and the ingredient M4 that are question targets, a question asking what the dish D1, the ingredient M1, the ingredient M2, or the ingredient M4 is.

For example, the question creation unit 103 creates a question asking “What is that dish?” for the dish D1, for example. Furthermore, the question creation unit 103 creates a question asking “What is that ingredient?” for the ingredient M1, the ingredient M2, and the ingredient M4, for example.

The question creation unit 103 outputs the question information to the action detection unit 105, the timing determination unit 107, and the question output unit 108.

Here, the question creation unit 103 creates, for example, question information in which information indicating the dish D1 is associated with “What is that dish?”, question information in which the ingredient M1 is associated with “What is that dish?”, question information in which the ingredient M2 is associated with “What is that dish?”, and question information in which the ingredient M4 is associated with “What is that dish?”.

The question creation unit 103 gives priority orders to the dish D1, the ingredient M1, the ingredient M4, and the ingredient M2 in such a manner that, for example, the dish D1, the ingredient M1, the ingredient M4, and the ingredient M2 are arranged in this order when they are arranged in descending order of priority.

The second image acquiring unit 104 acquires the second captured image captured by the second camera 22, and outputs the acquired second captured image to the action detection unit 105.

The action detection unit 105 detects an action of the user related to the question target on the basis of the second captured image acquired by the second image acquiring unit 104 (step ST4).

The action detection unit 105 detects, for example, a user's action of touching a tableware on which the dish D1, the ingredient M1, the ingredient M2, or the ingredient M4 is served, a user's action of holding the dish D1, the ingredient M1, the ingredient M2, or the ingredient M4 with cutlery, a user's action of putting the dish D1, the ingredient M1, the ingredient M2, or the ingredient M4 into his/her mouth, a user's action of chewing the dish D1, the ingredient M1, the ingredient M2, or the ingredient M4, and a user's action of swallowing the dish D1, the ingredient M1, the ingredient M2, or the ingredient M4.

The action detection unit 105 outputs action information regarding the detected action of the user to the timing determination unit 107.

The timing determination unit 107 determines a question timing at which the question created by the question creation unit 103 is output from the period in which the action detection unit 105 detects the user's action related to the question target in step ST4 (step ST5). Specifically, the timing determination unit 107 determines a time at which the user is performing an action related to the question target as the question timing.

Here, the timing determination unit 107 determines the time at which the user is performing an action related to the dish D1, the ingredient M1, the ingredient M2, or the ingredient M4 as the question timing.

Here, there are now multiple question targets (dish D1, ingredient M1, ingredient M2, and ingredient M4). Suppose that the priority order assigned to the dish D1 is the highest in the question information. In this case, the timing determination unit 107 determines the time at which the user is performing an action related to the dish D1 as the question timing. For example, the timing determination unit 107 determines, as the question timing, a time at which the user is touching a tableware on which the dish D1 is served, a time at which the user is holding the dish D1 with cutlery, a time at which the user is putting the dish D1 into his/her mouth, a time at which the user is chewing the dish D1, or a time at which the user is swallowing the dish D1.

When not determining the question timing (“NO” in step ST5), the timing determination unit 107 gives a notice indicating that the question timing has not been determined to the action detection unit 105. When receiving the notice indicating that the question timing has not been determined, the action detection unit 105 continuously detects an action of the user related to the question target on the basis of the second captured image acquired by the second image acquiring unit 104 (step ST4).

When determining the arrival of the question timing (“YES” in step ST5), the timing determination unit 107 outputs the timing arrival information to the question output unit 108.

Here, the timing determination unit 107 outputs, to the question output unit 108, timing arrival information indicating the arrival of the question timing for the dish D1.

The question output unit 108 outputs the question voice output information to the speaker 24 on the basis of the question information output from the question creation unit 103 in step ST3 and the timing arrival information output from the timing determination unit 107 in step ST5 (step ST6).

Here, the question output unit 108 creates question voice output information for outputting, for example, “What is that dish?” associated with the dish D1 in the question information by voice and outputs the created question voice output information to the speaker 24. As a result, a voice asking “What is that dish?” is output from the speaker 24.

When the user answers the question output in step ST6, the speech acquisition unit 109 waits until acquiring the uttered speech of the user answering the question (“NO” in step ST7).

When acquiring the uttered speech (“YES” in step ST7), the speech acquisition unit 109 outputs the acquired uttered speech to the speech recognition unit 110.

Note that the speech acquisition unit 109 constantly acquires surrounding uttered speeches. In step ST7, the speech acquisition unit 109 regards, for example, an uttered speech acquired immediately after the question output unit 108 outputs the question voice output information as the uttered speech of the user answering the question. For example, when outputting the question voice output information, the question output unit 108 gives a notice indicating the output of the question voice output information to the speech acquisition unit 109. Note that, in FIG. 1, an arrow from the question output unit 108 to the speech acquisition unit 109 is not illustrated.

The speech recognition unit 110 executes speech recognition processing and performs speech recognition on the uttered speech acquired by the speech acquisition unit 109 in step ST7 (step ST8).

The speech recognition unit 110 outputs the speech recognition result to the reflection unit 111.

The reflection unit 111 reflects, in the dietary intake information stored in the storage unit 106, information regarding a dish or an ingredient which has been specified on the basis of the speech recognition result by the speech recognition unit 110 in step ST8 and which has been obtained from the user answering the question output by voice by the question output unit 108 on the basis of the question voice output information by the question output unit 108 in step ST6 (step ST9). When doing so, the reflection unit 111 also sets the answer flag attached to the corresponding information indicating the dish or ingredient in the dietary intake information to “1”.

For example, it is assumed that the user has answered “beef curry” to the question asking “What is that dish?”. In this case, the reflection unit 111 reflects the answer in the dietary intake information. Furthermore, the reflection unit 111 also sets the answer flag attached to the information indicating the dish D1 in the dietary intake information to “1”. As a result, the reflection unit 111 updates the information regarding the dish D1 to the contents as illustrated in FIG. 7 in the dietary intake information stored in the storage unit 106.

When the process of step ST9 is completed, the operation of the dietary intake information acquisition device 1 returns to the process of step ST1.

The dietary content inferring unit 102 infers again a dish or an ingredient to be consumed by the user on the basis of the first captured image acquired by the first image acquiring unit 101 (step ST1).

The question creation unit 103 determines, on the basis of the dietary intake information in which the user's answer has been reflected by the reflection unit 111 in step ST9, whether or not there is a target dish or a target ingredient among the dishes or ingredients inferred by the dietary content inferring unit 102 in step ST2. That is, the question creation unit 103 determines again whether or not there is a question target among the dishes or ingredients inferred by the dietary content inferring unit 102 on the basis of the dietary intake information in which the information regarding the dish or ingredient answered by the user has been reflected by the reflection unit 111.

In this manner, the dietary intake information acquisition device 1 can improve the accuracy of the dietary intake information by repeating, for example, a question by interaction with the user as to what a dish or ingredient having low degree of certainty is and reflection of the answer obtained from the user in the dietary intake information.

Note that, when inferring a new ingredient included in a dish for which the dietary intake information has already been created in step ST1, the dietary content inferring unit 102 adds the newly inferred ingredient to the ingredients associated with the dish.

For example, it is assumed that dietary intake information having contents as illustrated in FIG. 7 is now stored in the storage unit 106.

It is assumed that, as a result of inferring again the dish or ingredient to be consumed by the user on the basis of the latest first captured image, the dietary content inferring unit 102 newly detects an ingredient M7 as an ingredient of the roux of curry and infers the detected ingredient M7 as eggplant. In this case, the dietary content inferring unit 102 adds information indicating eggplant to the information indicating the ingredient corresponding to the dish D1 in the dietary intake information.

As the user keeps eating, the state of the dish or the ingredient on the tableware changes. For example, an amount of food in the tableware decreases. Accordingly, there is a possibility that an ingredient that has not been detected by the image recognition processing for the first captured image until then is newly recognized.

Furthermore, for example, in a case where the dietary content inferring unit 102 infers a dish or an ingredient different from the dish or the ingredient inferred in the past with respect to the dish or the ingredient for which the dietary intake information has already been created in step ST1, the dietary content inferring unit 102 may update the dietary intake information to have a newly inferred content.

It is to be noted that the dietary content inferring unit 102 does not update the information regarding the dish or the ingredient to which the answer flag “1” is given in the dietary intake information. This is because the answer obtained from the user is expected to be reliable.

As described above, the dietary intake information acquisition device 1 infers a dish or an ingredient to be consumed by the user on the basis of a captured image and creates dietary intake information. The dietary intake information acquisition device 1 creates a question asking what a question target (target dish or target ingredient) is for the question target (target dish or target ingredient) among the inferred dishes or ingredients. Furthermore, the dietary intake information acquisition device 1 detects a user's action related to the question target (target dish or target ingredient) on the basis of the captured image.

Then, the dietary intake information acquisition device 1 determines a question timing at which the question is output from the period in which the user's action related to the question target (target dish or target ingredient) is detected. The dietary intake information acquisition device 1 outputs question voice output information for outputting the question by voice at the question timing, and when acquiring an uttered speech of the user answering the question, the dietary intake information acquisition device 1 reflects, in the dietary intake information, information regarding a dish or an ingredient which is specified on the basis of the speech recognition result of the uttered speech and which is obtained from the user answering the question.

The dietary intake information acquisition device 1 determines the question timing from the period in which the user's action related to the question target (target dish or target ingredient) is detected, whereby the user can intuitively identify the dish or ingredient that is being asked and answer without hesitation. As a result, the dietary intake information acquisition device 1 can reduce an occurrence of a situation in which the user does not know which dish or ingredient is specifically being asked and hesitates what to answer. That is, the dietary intake information acquisition device 1 can obtain information regarding the dish or the ingredient consumed by the user by asking the user a question in such a manner that the user can surely answer the consumed dish or ingredient.

As described above, the dietary intake information acquisition device 1 can easily and accurately acquire the dietary intake information for identifying the dietary status of the user.

In the first embodiment described above, in a case where there are multiple question targets, the timing determination unit 107 in the dietary intake information acquisition device 1 specifies which question target is selected as a question target to which the action performed by the user at a time to be determined as the question timing is related in accordance with the priority orders assigned to the question targets, but this is merely an example. The timing determination unit 107 may determine a time at which an action related to any one of the multiple question targets is detected as the question timing regardless of priority orders. In this case, the question creation unit 103 does not necessarily give priority orders to the question targets.

Furthermore, in the first embodiment described above, the question creation unit 103 may give a higher priority order to a target dish or a target ingredient that is obtained as an answer to a question and that can also be an answer to a question to another target dish or target ingredient.

In the above specific example, it is assumed that the dietary intake information has, for example, the contents as illustrated in FIG. 6. In this case, the question creation unit 103 determines that the dish D1 is the target dish and the ingredients M1, M2, and M4 are the target ingredients. Here, if it is determined that the ingredient M1 is beef, the question creation unit 103 can infer that the dish D1 is beef curry even if a question asking what the dish D1 is not output to the user.

Therefore, the question creation unit 103 gives a higher priority order to the ingredient M1. The question creation unit 103 may give the highest priority order to the ingredient M1 among the question targets, or may give a higher priority order than that of at least the dish D1. In this case, the question creation unit 103 adds, to the ingredient M1, information indicating that an answer to a question asking what the ingredient M1 is will also be an answer to the question to the dish D1. This information indicating that the answer to the question asking what the ingredient M1 is will also be an answer to the question to the dish D1 is passed to the reflection unit 111 via the question output unit 108, for example. The reflection unit 111 also updates information regarding the dish D1 when reflecting an answer to a question asking what the ingredient M1 is in the dietary intake information. For example, if an answer indicating “beef” is obtained from the user, the reflection unit 111 updates the information indicating the ingredient M1 to the information indicating beef and updates the information indicating the dish D1 to information indicating beef curry in the dietary intake information. Note that the reflection unit 111 may also set the degree of certainty of the ingredient M1 and the degree of certainty of the dish D1 to 100(%).

Note that information (hereinafter referred to as “multiple-answers-possible information”) that defines a dish or ingredient which is obtained as an answer to a question and which can also be an answer to a question for another dish or ingredient and the other dish or ingredient in that case is defined in advance on a dish basis by the administrator or the like. When creating the dish identification information, for example, the administrator or the like also creates the multiple-answers-possible information.

Furthermore, in the first embodiment described above, the question creation unit 103 in the dietary intake information acquisition device 1 determines that there is a target dish or a target ingredient in a case where there is a dish or an ingredient whose given degree of certainty is less than the question necessity determination threshold, but this is merely an example.

The question creation unit 103 can determine whether or not there is a target dish or a target ingredient in accordance with a condition according to the needs. For example, in a case where there is a dish or an ingredient whose degree of certainty is equal to or greater than a threshold, the question creation unit 103 may determine the dish or the ingredient as the target dish or the target ingredient.

In this case, the question creation unit 103 may create a question asking what the target dish or the target ingredient is by a question confirming the target dish or the target ingredient, such as “Are you sure that the dish is beef curry?” or “Are you sure that the ingredient is carrot?”.

In addition, in the first embodiment described above, the question creation unit 103 creates a question asking what the question target is using a demonstrative, but this is merely an example.

The question creation unit 103 may create a question that does not use a demonstrative. For example, the question creation unit 103 may create a question asking what the question target is without using a demonstrative, such as “What are you eating now?”, “Are you eating chicken curry?”, or “You are eating carrot, right?”. Note that the dietary intake information acquisition device 1 can obtain an answer as to what the question target is by natural conversation by creating a question asking what the question target is using a demonstrative rather than by creating a question not using a demonstrative. In addition, the dietary intake information acquisition device 1 can communicate with the user in a short sentence.

Furthermore, in the first embodiment described above, the question creation unit 103 may not have the function of determining the target dish or the target ingredient from among the dishes or ingredients inferred by the dietary content inferring unit 102, and may determine all the dishes or ingredients inferred by the dietary content inferring unit 102 as the target dish or the target ingredient.

In this case, the process of step ST2 can be skipped in the operation of the dietary intake information acquisition device 1 described with reference to the flowchart of FIG. 8.

In addition, in the first embodiment described above, in a case where the dietary content inferring unit 102 uses a machine learning model to obtain the inference result of the dish or ingredient to be consumed by the user and the degree of certainty of the inference result, the dietary intake information acquisition device 1 may include a training unit (not illustrated) that retrains the machine learning model when acquiring the uttered speech of the user answering the question.

For example, the training unit acquires a notice indicating that the dietary intake information has been updated from the reflection unit 111. The training unit also acquires the first captured image from the question creation unit 103 via the question output unit 108 and the reflection unit 111. The training unit creates training data based on the first captured image and the dietary intake information, and retrains the machine learning model.

In the first embodiment described above, a case in which the dietary intake information acquisition device 1 determines that there is the target dish and the target ingredient has been described as a specific example in the description of the configuration and operation of the dietary intake information acquisition device 1, but this is merely an example.

The dietary intake information acquisition device 1 may determine that there is only the target dish.

For example, it is assumed that the first captured image is a captured image obtained by capturing the dish D1 as illustrated in FIG. 4, but almost the entire dish is covered with brown roux, and only the brown roux can be recognized in the image recognition processing on the first captured image. In this case, the dietary content inferring unit 102 in the dietary intake information acquisition device 1 infers only an ingredient that is brown roux. The dietary content inferring unit 102 cannot infer whether the dish D1 is, for example, curry and rice or beef stew only from the brown roux. Therefore, the dietary content inferring unit 102 infers the dish D1 by referring to the dish identification information (see, for example, FIG. 5). The dietary content inferring unit 102 cannot find whether the dish D1 is curry (for example, beef curry or chicken curry) or beef stew even by referring to the dish identification information, and thus, the dietary content inferring unit 102 infers that the dish D1 is beef stew, for example, and sets the degree of certainty assigned to the information indicating the dish D1 to 30(%). In this case, the dish D1 is less than the question necessity determination threshold, and thus, the question creation unit 103 determines that the dish D1 is the target dish. In addition, the question creation unit 103 creates a question asking what the dish D1 is (for example, “What is that dish?”).

The action detection unit 105 detects a user's action related to the dish D1, for example, a user's action of touching a tableware on which the dish D1 is served, and the timing determination unit 107 determines a time at which the user's action of touching the tableware on which the dish D1 is served is detected by the action detection unit 105 as the question timing.

The question output unit 108 outputs the question voice output information to the speaker 24, and outputs by voice the question asking “What is that dish?” created by the question creation unit 103 from the speaker 24.

For example, it is assumed that the user has answered “curry” to the question asking “What is that dish?”. In this case, the reflection unit 111 updates information indicating beef stew for the dish D1 to the information indicating curry in the dietary intake information. However, even at this point, it is still not known whether the dish D1 is beef curry or chicken curry. Therefore, the reflection unit 111 infers that the dish D1 looks like beef curry, updates the information indicating beef stew to information indicating beef curry, and updates the degree of certainty assigned to the information indicating beef curry to 50(%) in the dietary intake information. Furthermore, the reflection unit 111 keeps “0” for the answer flag associated with the information indicating beef curry. As described above, in a case where a highly accurate answer cannot be obtained from the user by giving a question, the reflection unit 111 may consider that the information regarding the dish or the ingredient cannot be obtained from the user from the interaction with the user. Alternatively, the reflection unit 111 may set the answer flag to “1”, and set “1” to an additional-question necessity flag different from the answer flag in the dietary intake information. In a case where the additional-question necessity flag is set to “1”, the dietary content inferring unit 102 determines that the information indicating the dish or ingredient associated with the answer flag “1” can be updated even if the answer flag is “1”.

When the dietary intake information is updated to the information indicating beef curry for the dish D1 by the reflection unit 111, the question creation unit 103 again determines the dish D1 as a target dish and creates a question asking what the dish D1 is due to the degree of certainty attached to the information indicating beef curry being less than the question necessity determination threshold. Here, when creating a question asking what the dish D1 is for the second time, the question creation unit 103 may create a question asking directly whether or not the dish D1 is the dish D1 inferred by the dietary content inferring unit 102, for example, “Is that beef curry?”.

The question output unit 108 asks the user what the dish D1 is when the user's action of touching the tableware on which the dish D1 is served is detected again. For example, in a case where the user has answered “Yes” to the question of “Is that beef curry?”, the reflection unit 111 determines that the inference by the dietary content inferring unit 102 is correct, and then, keeps the information indicating beef curry unchanged and updates the degree of certainty assigned to the information indicating beef curry to 100(%) for the dish D1 in the dietary intake information. On the other hand, in a case where, for example, the user has answered “No”, the reflection unit 111 updates the information indicating beef curry to the information indicating chicken curry for the dish D1 and updates the degree of certainty assigned to the information indicating chicken curry to 70(%) in the dietary intake information, for example. The reflection unit 111 may output a question asking again what the dish D1 is with the information indicating beef curry being updated to information indicating chicken curry for the dish D1 and the degree of certainty assigned to the information indicating chicken curry being set as 50(%) in the dietary intake information.

Note that, as described above, when the user keeps eating, the dietary content inferring unit 102 in the dietary intake information acquisition device 1 may be able to infer the ingredient included in the dish D1 on the basis of the first captured image. Upon inferring the ingredients included in the dish D1, the dietary content inferring unit 102 adds information indicating the ingredients to the dietary intake information. The dietary content inferring unit 102 may infer that the dish D1 is beef curry by referring to the dish identification information when the dietary content inferring unit 102 can infer the ingredient included in the dish D1 as beef.

Further, the dietary intake information acquisition device 1 may determine that there is only the target ingredient.

For example, it is assumed that the dietary content inferring unit 102 can infer a rice ball on the basis of the first captured image, but cannot recognize an ingredient (ingredient X) in the rice ball by image recognition. In this case, the dietary content inferring unit 102 in the dietary intake information acquisition device 1 infers, from the first captured image, a rice ball that is a dish, dried seaweed that is an ingredient, and rice that is an ingredient, for example. Then, the dietary content inferring unit 102 infers the ingredient X by referring to the dish identification information. For example, the dietary content inferring unit 102 infers that the ingredient X is dried bonito flakes and calculates the degree of certainty given to the information indicating dried bonito flakes as 50(%). In this case, the degree of certainty of 50(%) is less than the question necessity determination threshold, and thus, the question creation unit 103 determines that the ingredient X is the target ingredient. In addition, the question creation unit 103 creates a question asking what the ingredient X is (for example, “What is that ingredient?”).

The action detection unit 105 detects a user's action related to the ingredient X, for example, a user's action of putting the ingredient X into his/her mouth, and the timing determination unit 107 determines a time at which the user's action of putting the ingredient X into his/her mouth is detected by the action detection unit 105 as the question timing.

The question output unit 108 outputs the question voice output information to the speaker 24, and outputs by voice the question asking “What is that ingredient?” created by the question creation unit 103 from the speaker 24.

Here, it is assumed, for example, that the user has answered “dried young sardines” to the question asking “What is that ingredient?”. In this case, the reflection unit 111 updates information indicating dried bonito flakes for the ingredient X to the information indicating dried young sardines in the dietary intake information. Furthermore, the reflection unit 111 updates the degree of certainty given to the information indicating dried young sardines to 100(%) in the dietary intake information. In addition, the reflection unit 111 updates the answer flag given to the information indicating dried young sardines to “1” in the dietary intake information.

In the first embodiment described above, the dietary intake information acquisition device 1 is mounted on the robot 2, but this is merely an example.

The dietary intake information acquisition device 1 may be mounted on, a device (not illustrated) having a voice output function such as a smart speaker.

In addition, the dietary intake information acquisition device 1 may be mounted on a server (not illustrated). For example, a part of the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111 may be mounted on the server.

Furthermore, in the first embodiment described above, the first camera 21 and the second camera 22 are separately provided, but this is merely an example. The first camera 21 and the second camera 22 may be a common camera.

Note that, in the first embodiment described above, the dietary intake information acquisition device 1 can be connected to a calorie calculation device (not illustrated) that calculates calories consumed by the user.

For example, for every update of the dietary intake information stored in the storage unit 106, the dietary intake information acquisition device 1 links the updated dietary intake information and the first captured image with the calorie calculation device. The calorie calculation device calculates the calories intaken by the user on the basis of the dietary intake information and the first captured image. Furthermore, the dietary intake information acquisition device 1 can link, for example, only the first captured image with the calorie calculation device, and the calorie calculation device can calculate the calories consumed by the user on the basis of the first captured image. The calorie calculation device may use a known technique of calculating intake calories based on the captured image to calculate the calories consumed by the user.

For example, information regarding the user's intake calories calculated by the calorie calculation device is used for recommendation or health management for improving the user's dietary life together with the dietary intake information created by the dietary intake information acquisition device 1.

Note that the dietary intake information acquisition device 1 may have a calorie calculation function.

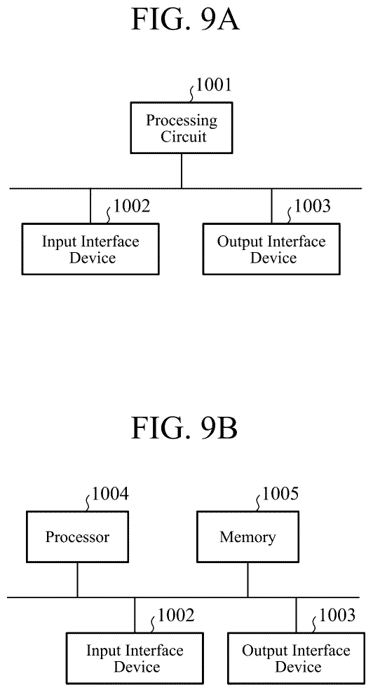

FIGS. 9A and 9B are diagrams illustrating an example of a hardware configuration of the dietary intake information acquisition device 1 according to the first embodiment.

In the first embodiment, functions of the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111 are implemented by a processing circuit 1001. That is, the dietary intake information acquisition device 1 includes the processing circuit 1001 for performing control to obtain information regarding the dish or the ingredient consumed by the user by asking the user a question in such a manner that the user can surely answer the consumed ingredient.

The processing circuit 1001 may be dedicated hardware as illustrated in FIG. 9A or a processor 1004 that executes a program stored in a memory as illustrated in FIG. 9B.

When the processing circuit 1001 is dedicated hardware, the processing circuit 1001 is, for example, a single circuit, a composite circuit, a programmed processor, a parallel programmed processor, an application specific integrated circuit (ASIC), a field-programmable gate array (FPGA), or a combination of some of these circuits.

In a case where the processing circuit is the processor 1004, the functions of the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111 are implemented by software, firmware, or a combination of software and firmware. Software or firmware is described as a program and stored in a memory 1005. The processor 1004 implements the functions of the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111 by reading and executing the program stored in the memory 1005. That is, the dietary intake information acquisition device 1 includes the memory 1005 for storing programs to eventually execute the steps ST1 to ST9 in FIG. 8 when being executed by the processor 1004. In addition, the program stored in the memory 1005 is considered to cause a computer to execute the procedures or methods performed by the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111. Here, the memory 1005 is, for example, a nonvolatile or volatile semiconductor memory such as RAM, read only memory (ROM), flash memory, erasable programmable read only memory (EPROM), or electrically erasable programmable read-only memory (EEPROM), a magnetic disk, a flexible disk, an optical disk, a compact disk, a mini disk, or a digital versatile disc (DVD).

Note that a portion of the functions of the first image acquiring unit 101, the dietary content inferring unit 102, the question creation unit 103, the second image acquiring unit 104, the action detection unit 105, the timing determination unit 107, the question output unit 108, the speech acquisition unit 109, the speech recognition unit 110, and the reflection unit 111 may be implemented by dedicated hardware, and another portion may be implemented by software or firmware. For example, the functions of the first image acquiring unit 101, the second image acquiring unit 104, and the speech acquisition unit 109 may be implemented by the processing circuit 1001 as dedicated hardware, and the functions of the dietary content inferring unit 102, the question creation unit 103, the action detection unit 105, the timing determination unit 107, the question output unit 108, the speech recognition unit 110, and the reflection unit 111 may be implemented by the processor 1004 reading and executing the program stored in the memory 1005.

The storage unit 106 includes the memory 1005, a hard disk drive (HDD), or the like.

In addition, the dietary intake information acquisition device 1 includes an input interface device 1002 and an output interface device 1003 that perform wired communication or wireless communication with devices such as the first camera 21, the second camera 22, the microphone 23, and the speaker 24.

As described above, according to the first embodiment, the dietary intake information acquisition device 1 includes: a dietary content inferring unit 102 to infer a dish or an ingredient to be consumed by a user on the basis of a captured image and create dietary intake information regarding the dish or the ingredient that has been inferred; a question creation unit 103 to create, for a target dish or a target ingredient among the dish or the ingredient inferred by the dietary content inferring unit 102, a question asking what the target dish or the target ingredient is; an action detection unit 105 to detect an action of the user related to the target dish or the target ingredient on the basis of the captured image; a timing determination unit 107 to determine a question timing at which the question created by the question creation unit 103 is output from a period in which the action detection unit 105 detects an action of the user related to the target dish or the target ingredient; a question output unit 108 to output question voice output information for outputting the question created by the question creation unit 103 by voice at the question timing determined by the timing determination unit 107; a speech acquisition unit 109 to acquire an uttered speech of the user in response to the question output by voice by the question output unit 108 on the basis of the question voice output information; a speech recognition unit 110 to perform speech recognition on the uttered speech acquired by the speech acquisition unit 109; and a reflection unit 111 to reflect, in the dietary intake information, information regarding the dish or the ingredient which has been obtained from the user answering the question and which has been specified on the basis of a speech recognition result by the speech recognition unit 110. The dietary intake information acquisition device 1 determines the question timing from the period in which a user's action related to a question target (target dish or target ingredient) is detected, whereby the user can intuitively identify the dish or ingredient that is being asked and answer without hesitation. As a result, the dietary intake information acquisition device 1 can reduce an occurrence of a situation in which the user does not know which dish or ingredient is specifically being asked and hesitates what to answer. That is, the dietary intake information acquisition device 1 can obtain information regarding the dish or the ingredient consumed by the user by asking the user a question in such a manner that the user can surely answer the consumed ingredient.

In addition, the dietary intake information acquisition device 1 can be configured in such a manner that the question creation unit 103 determines whether or not there is the dish or the ingredient that is a target to which the question is to be output among the dish or the ingredient inferred by the dietary content inferring unit 102 on the basis of the dietary intake information created by the dietary content inferring unit 102, and creates the question with the dish or the ingredient determined to be the target to which the question is to be output as the target dish or the target ingredient. The dietary intake information acquisition device 1 can reduce a burden on the user of answering a question unnecessarily asked to the user by narrowing down the target dish or the target ingredient.

In addition, the dietary intake information acquisition device 1 can be configured in such a manner that the timing determination unit 107 determines whether or not there is a speech on the basis of the speech recognition result by the speech recognition unit 110, and does not determine the question timing while there is the speech. With this configuration, the dietary intake information acquisition device 1 can prevent an output of a question asking the user what the target dish or the target ingredient is in a situation where the user is hard to hear by speech of the user or the like.

In addition, the dietary intake information acquisition device 1 can be configured in such a manner that the question creation unit 103 creates the question asking what the target dish or the target ingredient is, using a demonstrative. With this configuration, the dietary intake information acquisition device 1 can obtain an answer as to what the question target is by natural conversation. In addition, the dietary intake information acquisition device 1 can communicate with the user in a short sentence.

In addition, the dietary intake information acquisition device 1 can be configured in such a manner that the dietary content inferring unit 102 calculates degree of certainty indicating certainty of an inference result of the dish or the ingredient, and the question creation unit 103 determines whether or not there is the target dish or the target ingredient among the dish or the ingredient inferred by the dietary content inferring unit 102 on the basis of the degree of certainty calculated by the dietary content inferring unit 102. For example, the question creation unit 103 determines that there is a target dish or a target ingredient in a case where there is a dish or an ingredient whose degree of certainty is less than the question necessity determination threshold, by which the dietary intake information acquisition device 1 narrows down the target dish or the target ingredient. Therefore, the dietary intake information acquisition device 1 can reduce a burden on the user of answering a question unnecessarily asked to the user.

In addition, the dietary intake information acquisition device 1 can be configured in such a manner that the question creation unit 103 determines again whether or not there is a target dish or a target ingredient among the dish or ingredient inferred by the dietary content inferring unit 102 on the basis of the dietary intake information in which information regarding the dish or the ingredient obtained as an answer from the user has been reflected by the reflection unit 111. With this configuration, the dietary intake information acquisition device 1 can improve the accuracy of the dietary intake information by repeating a question by interaction with the user as to what the target dish or the target ingredient is and reflection of the answer obtained from the user in the dietary intake information.

In addition, the dietary intake information acquisition device 1 can be configured in such a manner that the question creation unit 103 gives, when there is a plurality of the target dishes or the target ingredients, priority orders to the plurality of the target dishes or the target ingredients, and the timing determination unit 107 determines the question timing in accordance with the priority orders. With this configuration, the dietary intake information acquisition device 1 can efficiently ask the user what the target dish or the target ingredient is.

In addition, the dietary intake information acquisition device 1 can be configured in such a manner that the question creation unit 103 gives a higher priority order to the target dish or the target ingredient that is obtained as an answer to the question and that is also possibly an answer to a question to another target dish or target ingredient, and the timing determination unit 107 determines the question timing in accordance with the priority orders. With this configuration, the dietary intake information acquisition device 1 can obtain an answer as to what the plurality of target dishes or target ingredients is with a small number of questions to the user.

It is to be noted that any components in the embodiment can be modified or omitted.

Various aspects of the present disclosure will be collectively described below as supplementary matters.

### Supplementary Matter 1

A dietary intake information acquisition device including:
a dietary content inferring unit to infer a dish or an ingredient to be consumed by a user on the basis of a captured image and create dietary intake information regarding the dish or the ingredient that has been inferred;
a question creation unit to create, for a target dish or a target ingredient among the dish or the ingredient inferred by the dietary content inferring unit, a question asking what the target dish or the target ingredient is;
an action detection unit to detect an action of the user related to the target dish or the target ingredient on the basis of the captured image;
a timing determination unit to determine a question timing at which the question created by the question creation unit is output from a period in which the action detection unit detects an action of the user related to the target dish or the target ingredient;
a question output unit to output question voice output information for outputting the question created by the question creation unit by voice at the question timing determined by the timing determination unit;
a speech acquisition unit to acquire an uttered speech of the user in response to the question output by the question output unit by voice on the basis of the question voice output information;
a speech recognition unit to perform speech recognition on the uttered speech acquired by the speech acquisition unit; and
a reflection unit to reflect, in the dietary intake information, information regarding the dish or the ingredient which has been obtained from the user answering the question and which has been specified on the basis of a speech recognition result by the speech recognition unit.

### Supplementary Matter 2

The dietary intake information acquisition device according to Supplementary Matter 1, in which
the question creation unit determines whether or not there is the dish or the ingredient that is a target to which the question is to be output among the dish or the ingredient inferred by the dietary content inferring unit on the basis of the dietary intake information created by the dietary content inferring unit, and creates the question with the dish or the ingredient determined to be the target to which the question is to be output as the target dish or the target ingredient.

### Supplementary Matter 3

The dietary intake information acquisition device according to Supplementary Matter 1 or 2, in which
the action detection unit detects an action of the user of touching a tableware on which the target dish or the target ingredient is served.

### Supplementary Matter 4

The dietary intake information acquisition device according to any one of Supplementary Matters 1 to 3, in which
the action detection unit detects an action of the user of holding the target dish or the target ingredient with cutlery.

### Supplementary Matter 5

The dietary intake information acquisition device according to any one of Supplementary Matters 1 to 4, in which
the action detection unit detects an action of the user of putting the target dish or the target ingredient into a mouth of the user or an action of the user of chewing the target dish or the target ingredient.

### Supplementary Matter 6

The dietary intake information acquisition device according to any one of Supplementary Matters 1 to 5, in which
the action detection unit detects an action of the user of swallowing the target dish or the target ingredient.

### Supplementary Matter 7

The dietary intake information acquisition device according to any one of Supplementary Matters 1 to 6, in which
the timing determination unit determines a period during which the user continues the action related to the target dish or the target ingredient as the question timing.

### Supplementary Matter 8

The dietary intake information acquisition device according to any one of Supplementary Matters 1 to 7, in which
the speech acquisition unit acquires the uttered speech of surroundings, and
the timing determination unit determines whether or not there is a speech on the basis of the speech recognition result by the speech recognition unit, and does not determine the question timing while there is the speech.

### Supplementary Matter 9

The dietary intake information acquisition device according to any one of Supplementary Matters 1 to 8, in which
the question creation unit creates the question asking what the target dish or the target ingredient is, using a demonstrative.

### Supplementary Matter 10

The dietary intake information acquisition device according to Supplementary Matter 2, in which
the dietary content inferring unit calculates degree of certainty indicating certainty of an inference result of the dish or the ingredient, and
the question creation unit determines whether or not there is the target dish or the target ingredient among the dish or the ingredient inferred by the dietary content inferring unit on the basis of the degree of certainty calculated by the dietary content inferring unit.

### Supplementary Matter 11

The dietary intake information acquisition device according to Supplementary Matter 2, in which
the question creation unit determines again whether or not there is the target dish or the target ingredient among the dish or the ingredient inferred by the dietary content inferring unit on the basis of the dietary intake information in which information regarding the dish or the ingredient obtained from the user as an answer has been reflected by the reflection unit.

### Supplementary Matter 12

The dietary intake information acquisition device according to any one of Supplementary Matters 1 to 11, in which
the question creation unit gives, when there is a plurality of the target dishes or the target ingredients, priority orders to the plurality of the target dishes or the target ingredients, and
the timing determination unit determines the question timing in accordance with the priority orders.

### Supplementary Matter 13

The dietary intake information acquisition device according to Supplementary Matter 12, in which
the question creation unit gives a higher priority order to the target dish or the target ingredient that is obtained as an answer to the question and that is also possibly an answer to a question for another target dish included in the target dish or another target ingredient included in the target ingredient.

### Supplementary Matter 14

A dietary intake information acquisition method including:
inferring a dish or an ingredient to be consumed by a user on the basis of a captured image and creating dietary intake information regarding the dish or the ingredient that has been inferred, using a dietary content inferring unit;
creating, for a target dish or a target ingredient among the dish or the ingredient inferred by the dietary content inferring unit, a question asking what the target dish or the target ingredient is, using a question creation unit;
detecting an action of the user related to the target dish or the target ingredient on the basis of the captured image using an action detection unit;
determining, using a timing determination unit, a question timing at which the question created by the question creation unit is output from a period in which the action detection unit detects an action of the user related to the target dish or the target ingredient;
outputting question voice output information for outputting the question created by the question creation unit by voice at the question timing determined by the timing determination unit, using a question output unit;
acquiring an uttered speech of the user in response to the question output by the question output unit by voice on the basis of the question voice output information, using a speech acquisition unit;
performing speech recognition on the uttered speech acquired by the speech acquisition unit using a speech recognition unit; and
reflecting, in the dietary intake information, information regarding the dish or the ingredient which has been obtained from the user answering the question and which has been specified on the basis of a speech recognition result by the speech recognition unit, using a reflection unit.

### REFERENCE SIGNS LIST

1: dietary intake information acquisition device, 101: first image acquiring unit, 102: dietary content inferring unit, 103: question creation unit, 104: second image acquiring unit, 105: action detection unit, 106: storage unit, 107: timing determination unit, 108: question output unit, 109: speech acquisition unit, 110: speech recognition unit, 111: reflection unit, 2: robot, 21: first camera, 22: second camera, 23: microphone, 24: speaker, 25: drive device, 201: drive control unit, 1001: processing circuit, 1002: input interface device, 1003: output interface device, 1004: processor, 1005: memory

1. A dietary intake information acquisition device comprising processing circuitry
to perform inferring of a dish or an ingredient to be consumed by a user on a basis of a captured image and perform creation of dietary intake information regarding the dish or the ingredient that has been inferred,
to perform creation of, for a target dish or a target ingredient among the dish or the ingredient inferred by the inferring, a question asking what the target dish or the target ingredient is,
to perform detection of an action of the user related to the target dish or the target ingredient on a basis of the captured image,
to perform determination of a question timing at which the question created by the creation is output from a period in which an action of the user related to the target dish or the target ingredient is detected by the detection,
to perform output of question voice output information for outputting the question created by the creation of the question by voice at the question timing determined by the determination,
to perform acquisition of an uttered speech of the user in response to the question output by the output by voice on a basis of the question voice output information,
to perform speech recognition on the uttered speech acquired by the acquisition, and
perform reflection of, in the dietary intake information, information regarding the dish or the ingredient which has been obtained from the user performing answer of the question and which has been specified on a basis of a result of the speech recognition.

2. The dietary intake information acquisition device according to claim 1, wherein the processing circuitry determines whether or not there is the dish or the
ingredient that is a target to which the question is to be output among the dish or the ingredient inferred by the inferring on a basis of the dietary intake information created by the creation of the dietary intake information, and creates the question with the dish or the ingredient determined to be the target to which the question is to be output as the target dish or the target ingredient.

3. The dietary intake information acquisition device according to claim 1, wherein
the processing circuitry detects an action of the user of touching a tableware on which the target dish or the target ingredient is served.

4. The dietary intake information acquisition device according to claim 1, wherein
the processing circuitry detects an action of the user of holding the target dish or the target ingredient with cutlery.

5. The dietary intake information acquisition device according to claim 1, wherein
the processing circuitry detects an action of the user of putting the target dish or the target ingredient into a mouth of the user or an action of the user of chewing the target dish or the target ingredient.

6. The dietary intake information acquisition device according to claim 1, wherein
the processing circuitry detects an action of the user of swallowing the dish or the target ingredient.

7. The dietary intake information acquisition device according to claim 1, wherein
the processing circuitry determines a period during which the user continues the action of the user related to the target dish or the target ingredient as the question timing.

8. The dietary intake information acquisition device according to claim 1, wherein
the processing circuitry acquires the uttered speech of surroundings, and
the processing circuitry determines whether or not there is a speech on a basis of the result of the speech recognition, and does not determine the question timing while there is the speech.

9. The dietary intake information acquisition device according to claim 1, wherein
the processing circuitry creates the question asking what the target dish or the target ingredient is, using a demonstrative.

10. The dietary intake information acquisition device according to claim 2, wherein
the processing circuitry calculates degree of certainty indicating certainty of an inference result of the dish or the ingredient, and
the processing circuitry determines whether or not there is the target dish or the target ingredient among the dish or the ingredient inferred by the inferring on a basis of the degree of certainty.

11. The dietary intake information acquisition device according to claim 2, wherein
the processing circuitry determines again whether or not there is the target dish or the target ingredient among the dish or the ingredient inferred by the inferring on a basis of the dietary intake information in which information regarding the dish or the ingredient obtained from the user as the answer has been reflected by the reflection.

12. The dietary intake information acquisition device according to claim 1, wherein
the processing circuitry gives, when there is a plurality of the target dishes or the target ingredients, priority orders to the plurality of the target dishes or the target ingredients, and
the processing circuitry determines the question timing in accordance with the priority orders.

13. The dietary intake information acquisition device according to claim 12, wherein
the processing circuitry gives a higher priority order to the target dish or the target ingredient that is obtained as an answer to the question and that is also possibly an answer to a question for another target dish included in the target dish or another target ingredient included in the target ingredient.

14. A dietary intake information acquisition method comprising:
perform inferring of a dish or an ingredient to be consumed by a user on a basis of a captured image and perform creation of dietary intake information regarding the dish or the ingredient that has been inferred;
to perform creation of, for a target dish or a target ingredient among the dish or the ingredient inferred by the inferring, a question asking what the target dish or the target ingredient is;
to perform detection of an action of the user related to the target dish or the target ingredient on a basis of the captured image;
to perform determination of a question timing at which the question created by the creation is output from a period in which an action of the user related to the target dish or the target ingredient is detected by the detection;
to perform output of question voice output information for outputting the question created by the creation of the question by voice at the question timing determined by the determination;
to perform acquisition of an uttered speech of the user in response to the question output by the output by voice on a basis of the question voice output information;
performing speech recognition on the uttered speech acquired by the acquisition; and
performing reflection of, in the dietary intake information, information regarding the dish or the ingredient which has been obtained from the user performing answer of the question and which has been specified on a basis of a result of the speech recognition.
